(19) United States
(12) Patent Application Publication
(10) Pub. No.: US 2011/0301249 A1
(43) Pub. Date: Dec. 8, 2011

(54) STEM CELL ENHANCED PROTEIN PRODUCTS AND USES THEROF

(76) Inventor: Kedar Challakere, Atascadero, CA (US)

(21) Appl. No.: 13/133,786

(22) PCT Filed: Dec. 11, 2009
(86) PCT No.: PCT/US2009/067721

§ 371 Date: Aug. 26, 2011

Related U.S. Application Data
(60) Provisional application No. 61/121,990, filed on Dec. 12, 2008.

(51) Int. Cl. A61K 47/36; A23L 1/30; A23K 1/16; A23L 1/304; A23L 1/305; A61K 47/42; A23L 1/302

(52) U.S. Cl. 514/777; 514/783; 426/61; 426/2

(57) ABSTRACT

Methods for producing in-vitro cultured protein products that are enhanced with stem cells are disclosed. In-vitro cultured protein product compositions produced by said methods are also disclosed. The present invention also discloses methods of providing nutrients to an animal by feeding said animal with said in-vitro cultured protein products.

### RELATED APPLICATIONS

The present application claims priority to, and incorporates by reference, the entirety of U.S. Provisional Patent Application No. 61/121,990, filed on Dec. 12, 2008.

### TECHNICAL FIELD

The present invention relates to in-vitro produced protein products, such as food products for animal or human consumption that contain in-vitro cultured stem cells. The invention also relates to processes for producing and using said protein products.

### HISTORY OF RELATED ART

Meeting the global demand of meat consumption is an ongoing problem. In much of the world, meat consumption is rising steadily and is expected to double by the year 2050, as reported recently by the United Nations Food and Agricultural Organization. This increasing demand for meat and other sources of protein may no longer be sustained by traditional livestock production systems. To raise meat output, livestock producers have adopted new, intensive rearing techniques that rely on grains and legumes to feed their animals. In the face of world grain shortage, this method of producing meats from whole animals is highly inefficient because a significant portion of agriculturally produced grain is used for animal rather than human consumption. Additionally, the factory-style livestock industries, now firmly entrenched in industrial countries, have environmental side-effects ranging from growing the vast quantities of feed grain to disposing of manure. Furthermore, large livestock populations emit the potent greenhouse gas methane into the atmosphere, contributing to climate change. Hence, both meat production and consumption have adverse effects on the environment as well as animal welfare.

Moreover, the harmful effects of meat consumption on human health are well documented. For instance, it has been determined that over-consumption of animal fats is associated with an increased risk of cardiovascular disease, stroke and diabetes. Additionally, contaminated meat products are responsible for food borne diseases that are highly prevalent in the United States of America.

Thus, a need exists for increasing the availability of affordable protein products for human or animal consumption that minimize the use of animal farming and animal killing. A need also exists for producing such protein products with minimal environmental impact.

### SUMMARY

In view of the foregoing and other considerations, presented herein are methods of producing in-vitro cultured protein products enhanced with steprn cells that can be used as a nutrient source. In some embodiments, the methods comprise: (1) isolating stem cells from an organism; (2) culturing the stem cells in a growth medium; (3) attaching the stem cells to a scaffold; (4) inducing a migration of the stem cells onto the scaffold, such as a three dimensional edible scaffold; and (5) inducing a differentiation of the stem cells into a certain cell type. Additionally, disclosed are methods of customizing the in-vitro cultured stem cell enhanced protein products by adding to the growth medium various additives, such as bioproteins, vitamins, minerals, amino acids, ribonucleotides, nutrients, medicaments, prebiotics, probiotics, and the like.

Other embodiments of the present disclosure provide in-vitro cultured protein product compositions produced by the foregoing methods. In additional embodiments, methods are disclosed for providing nutrients to an animal by feeding the animal the in-vitro cultured protein products produced according to the aforementioned methods.

Various embodiments may provide one, some, or none of the above-listed benefits. Such aspects described herein are applicable to illustrative embodiments and it is noted that there are many and various embodiments that can be incorporated into the spirit and principles of the present invention. Accordingly, the above summary of the invention is not intended to represent each embodiment or every aspect of the present invention. Additional features and advantages of the invention will be described hereinafter which form the subject of the claims of the invention.

### BRIEF DESCRIPTION OF THE DRAWINGS

A more complete understanding of the methods and compositions of the present invention may be obtained by reference to the following Detailed Description when taken in conjunction with the accompanying Drawings, wherein:

FIG. 1 is a flow chart depicting a method of producing in-vitro cultured protein products in accordance with some embodiments of the present disclosure;

FIG. 2 is a depiction of a bioreactor that can be used to produce in-vitro cultured protein products in accordance with some embodiments of the present disclosure; and

FIG. 3 is a diagram depicting various steps to isolate stem cells from a Hydra species to produce in-vitro cultured protein products, in accordance with further embodiments of the present disclosure.

### DETAILED DESCRIPTION

Growing affluence and industrialization of meat production has led to an increase in the global demand for meat and protein products with the taste/texture known as umami. The assembly line meat factories consume enormous amounts of energy, pollute water supplies, generate significant green house gases, and require ever increasing dependency on grains that has led to the destruction of tropical rain forests. In the face of the tremendous environmental impact of current meat production methods, as well as an ever-increasing global demand for meat, novel production processes for stem cell enhanced protein products are disclosed herein. In various embodiments, the novel production processes disclosed herein can minimize environmental impact, reduce animal suffering, decrease danger to human health, reduce cost of protein production, and provide greater consumer choice. Additionally, novel stem cell enhanced protein products produced by the aforementioned processes are also disclosed.

Reference is now made in detail to illustrative embodiments of the invention as shown in the accompanying drawings. Wherever possible, the same reference numerals are used throughout the drawings to refer to the same or similar parts. The following discussion is presented to enable a person skilled in the art to make and use the invention. The general principles described herein may be applied to embodiments and applications other than those detailed below without departing from the spirit and scope of the disclosed invention as defined by the appended claims. Furthermore, the invention disclosed herein is not intended to be limited to the embodiments shown, but is to be accorded the widest scope consistent with the principles and features disclosed herein.

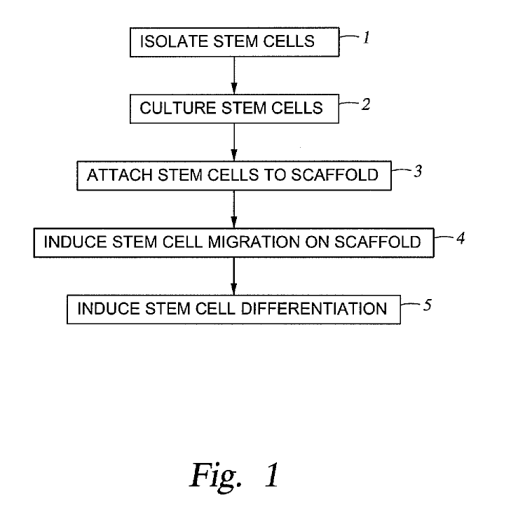

As depicted in the flow chart in FIG. 1, the present disclosure provides methods for producing in-vitro cultured protein products that are enhanced with stem cells. Such methods generally comprise isolating stem cells from an organism (step 1); culturing the stem cells in a growth medium (step 2); attaching the stem cells to a scaffold (step 3); inducing a migration of the stem cells onto the scaffold (step 4); and inducing a differentiation of the stem cells (step 5). In some embodiments, all of the above-mentioned steps may occur sequentially to produce the in-vitro cultured protein product compositions of the present disclosure. In other embodiments, one or more of the above-mentioned steps may be absent.

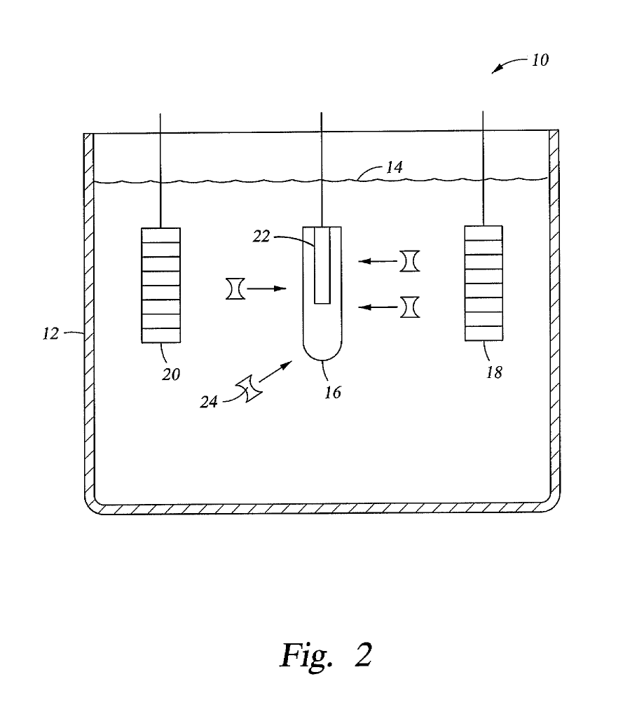

In some embodiments, one or more of the above-mentioned steps may occur in an apparatus, such as a bioreactor. Referring now to FIG. 2, bioreactor 10 is shown as an example of a bioreactor that may be suitable for facilitating the production of in-vitro cultured protein products of the present disclosure. In this embodiment, bioreactor 10 consists of a container 12 that houses growth medium 14, scaffold 16, and electrodes 18, 20, and 22. In some embodiments, electrodes 18 and 20, 20 and 22, and/or 18 and 22 may be of opposite polarity to each other.

In operation, isolated stem cells 24 may be cultured in growth medium 14 of bioreactor 10. Thereafter, cultured stem cells 24 attach onto and migrate on scaffold 16. As discussed in more detail below, such attachment and migration may be facilitated by agitating the growth medium. As also discussed in more detail below, the attachment and migration may be facilitated by current flow from electrodes 18, 20 and 22. The stem cells on scaffold 16 may then be induced to differentiate into one or more cell types by various methods that are discussed in more detail below (e.g., addition of differentiation-inducing agents to growth medium 14). In this embodiment, the resulting scaffold 16 that contains the differentiated stem cells constitutes the stem cell enhanced in-vitro cultured protein product.

As discussed in more detail below, various aspects of the present invention have numerous embodiments. Reference will now be made to the specific embodiments of the present disclosure for illustrative purposes only, and without limiting the scope of the present invention.

Isolation of Stem Cells

In general, stem cells may be isolated from any organism, such as organisms consumed by humans. Non-limiting examples of stem cell sources include, without limitation, mammals (e.g. cattle, buffalo, pigs, sheep, deer, etc.), birds (e.g. chicken, ducks, ostrich, turkey, pheasant, etc.), fish (e.g. swordfish, salmon, tuna, sea bass, trout, catfish, etc.), reptiles (e.g. snake, alligator, turtle, etc.), amphibians (e.g. frog legs), and invertebrates (e.g. lobster, crab, shrimp, clams, oysters, mussels, sea urchin, a Hydra species etc.).

In general, stem cells isolated from an organism may be derived from various cell lines and tissues. For instance, in some embodiments, stem cells may be derived from fibroblasts, myoblasts, epithelial stem cells, endothelial stem cells, interstitial cells, mesenchymal stem cells, hematopoietc stem cells, neural stem cells, and mesangioblasts. In more specific embodiments, the isolated stem cells may be pluri-potent embryonic mesenchymal stem cells that have the ability to differentiate into various cell lines, such as muscle cells, fat cells, bone cells, and/or cartilage cells. In further embodiments, the isolated stem cells may be totipotent embryonic stem cells, such as stem cells derived from the blastocyst stage of an embryo, fertilized eggs, placenta, or umbilical cords. Isolation of stem cells from additional sources can also be envisioned by a person of ordinary skill in the art.

In some embodiments, the isolation step only results in the isolation of the desired stem cells. In other embodiments, the isolation step results in the isolation of the desired stem cells as well as their niche aggregates. Niche aggregates generally refer to clusters of cells that are associated with a particular type of stem cell. In some embodiments, niche aggregates may consist of at least two different types of cells. In other embodiments, niche aggregates may consist of three different types of cells. In more specific embodiments, niche aggregates may include endothelial cells, epithelial cells, and/or interstitial cells.

Without being bound by theory, it is envisioned that niche aggregates facilitate the proliferation and/or differentiation of isolated stem cells. For instance, cells of the columnar body of Hydra species consist of epithelial and endothelial cells that are constantly in cell cycle. When cultured as homogenous groups of cells (e.g., endothelial or epithelial cells alone), it is envisioned that the isolated cells would not continue to proliferate. However, when the cells are cultured as heterogeneous cells that consist of various types of cells (e.g., endothelial, epithelial and interstitial cells), it is envisioned that the cell groups would proliferate more readily (i.e., undergo more cell division cycles). Accordingly, in some embodiments of the present disclosure, the isolation of stem cells and their niche aggregates from a Hydra species advantageously provides an ample source of stem cells that can be used in accordance with various embodiments of the present disclosure.

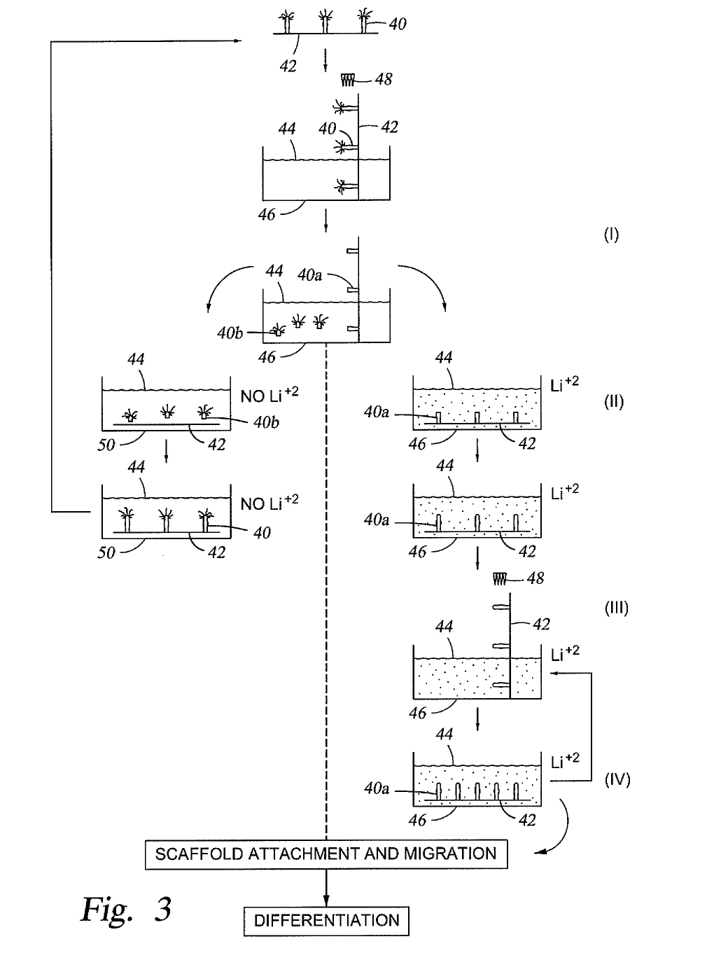

Stem cells (and their niche aggregates in some embodiments) may be isolated by various methods from numerous sources. For instance, in some embodiments, stem cells and their niche aggregates may be isolated from blood, plasma or muscle/organ biopsies. In some embodiments, stem cells and their niche aggregates may be isolated from the sectioning of an organism, such as the lower 30-90% of an organism with complete regenerative potential. In more specific embodiments that are depicted in FIG. 3 and discussed in more detail in Example 2 below, stem cells may be isolated from the sectioning of a Hydra species.

Culturing of Stem Cells

A person of ordinary skill in the art can envision that various methods may be used to culture stem cells after they are isolated. In some embodiments, growth media suitable for the growth of stem cells in vitro may be used to culture the isolated stem cells. As known by a person of ordinary skill in the art, such growth media can include one or more of the following components in various concentrations: protein, fat, fiber, moisture, vitamins (e.g., vitamins A, D, B, B₁₂, and E), choline, ascorbic acid, inositaol, niacin, pantothenic acid, phosphorous, lithium citrate, lithium chloride, and the like.

In more specific embodiments, a growth medium suitable for culturing stem cells may contain the following compositions known in the art: protein (e.g., about 45% to about 50% by weight, preferably from methanobacteria); fat (e.g., about 7% to about 15% by weight, preferably Omega-3 from algae); fiber (e.g., about 7% by weight); moisture (e.g., about 8% to about 10% by weight); vitamin A (e.g., about 8000 RJ/1, preferably from vegetarian sources); vitamin D (e.g., about 800 RJ/1, preferably from vegetarian sources); vitamin E (e.g., about 100 IU/l, preferably from vegetarian sources); choline (e.g., about 500 mg/l, preferably from vegetarian sources); ascorbic acid (e.g., about 200 mg/l, preferably from vegetarian sources); inositaol (e.g., about 100 mg/l, preferably from vegetarian sources); niacin (e.g., about 100 mg/l, preferably from vegetarian sources); pantothenic acid (e.g., about 80 mg/l, preferably from vegetarian sources); and phosphorous (e.g., about 0.5% to about 0.7% by weight, preferably from vegetarian sources).

In addition, the above-mentioned growth medium compositions may be varied in order to alter the membrane characteristics of the stem cells and their niche aggregates (e.g., an increase in fat %, addition of amino acids, etc.). Without being bound by theory, it is envisioned that varying the composition of the growth medium may change the glycoprotein/lipid composition of cell membranes. Applicant envisions that such variations to the growth medium can in turn improve the taste and umami of the protein product composition.

In some embodiments, dimethyl sulfoxide (DMSO) may also be added to a growth medium. Without being bound by theory, it is envisioned that DMSO helps facilitate absorption of nutrients by stem cells, thereby facilitating their growth. For instance, in some embodiments, DMSO may help epithelial cells regenerate cell clusters and cell cultures.

In further embodiments, the above-mentioned growth medium may also contain lithium citrate and/or lithium chloride. As discussed in more detail below, such additives can be particularly useful when stem cells are being derived directly from regenerative organisms, such as a Hydra species. In particular, and without being bound by theory, it is envisioned that lithium citrate and lithium chloride impede the regeneration process during stem cell isolation. For instance, lithium chloride and lithium acetate can help prevent the formation of a neural organizer, a neural complex and/or a head system in a Hydra species.

The growth media suitable for culturing stem cells in the present disclosure may also comprise one or more additives. In some embodiments, the one or more additives may be used to increase the nutritional value of the protein product to be produced. In some embodiments, the one or more additives may add nutrients that may not be present in conventional protein products. In related embodiments, the one or more additives may function to incorporate drugs or vitamins in the protein product. In yet other embodiments, the one or more additives may function to provide taste (umami) to the protein product. In more specific embodiments, the one or more additives may include, without limitation, bioproteins, vitamins, minerals, amino acids, ribonucleotides (e.g., inosinate and guanylate), nutrients, medicaments, prebiotics, probiotics, drugs and/or antigens.

The growth media suitable for culturing stem cells in the present disclosure may also consist of standard electolyte rich broths. In some embodiments, such broths contain proliferation factors to expand the pool of transit amplification cells. In some embodiments, such broths also contain amino acids generated from methanobacteria or from other non-animal sources. In addition, drugs, antigenic peptides, ribonucleotides, hormones, lipid carrier molecules (with or without bioactive agents), and pro-drugs may also be added to the broth.

The above-described growth media may be used in various containers or systems for culturing stem cells. For instance, in some embodiments, the above-described growth media may be used in bioreactors, such as bioreactor 10 shown in FIG. 2. Accordingly, in more specific embodiments, the above-described growth media may represent growth medium 14 in bioreactor 10 for culturing stem cells 24.

In general, bioreactors suitable for the present disclosure may be stationary, vibratory, or rotating. Applicants envision that bioreactors (such as bioreactor 10 in FIG. 2) can produce greater volume of cells while allowing greater control over the flow of nutrients, gases, metabolites, and regulatory molecules. Furthermore, bioreactors may provide physical and mechanical signals, such as compression, to stimulate cells. Such stimulations may lead to the production of specific biomolecules and/or stem cell differentiation.

Culture Conditions

The above-described growth media may also be used under different culture conditions. In some embodiments, stem cell culture conditions may include static, stirred, or dynamic flow conditions. Stem cells may also be grown at various temperatures and for various periods of time, as known by a person of ordinary skill in the art. In a preferred embodiment, the temperature range utilized will be between about 18 degrees Celsius to about 25 degrees Celsius. Likewise, in a preferred embodiment, the incubation times may vary from about three days to about fourteen days.

Attachment and Migration of Stem Cells onto Scaffolds

In general, scaffolds of the present disclosure provide structures for stem cells to attach to and migrate on. In some embodiments, such attachment and migration can be induced by exposing a scaffold to a growth medium that contains the cultured stem cells. In such embodiments, the attachment and migration of the stem cells onto the scaffold may also be induced by agitating the growth medium. The attachment and migration of the stem cells onto the scaffold in such embodiments may also be induced by applying an electrical or magnetic field to the growth medium.

In a more specific example, bioreactor 10 in FIG. 2 may be used to induce the attachment and migration of stem cells 24 onto scaffold 16. In some embodiments, this can occur through the agitation of growth medium 14. In some embodiments, electrodes 18, 20, and 22 may also be used to apply an electrical field to growth medium 14 in order to facilitate the attachment and migration steps. Other methods of inducing the attachment and migration of stem cells onto a scaffold can also be envisioned by persons of ordinary skill in the art.

Without being bound by theory, it is envisioned that scaffolds help add taste and texture to the in-vitro cultured protein products of the present disclosure by utilizing the membrane properties of the steprn cells, and by varying the density of the stem cells within the scaffold. Specifically, scaffolds may adjust the texture and taste (umami) of the in-vitro cultured protein products by varying the density of the stem cells that they harbor.

Various scaffold compositions may be used to help produce the in-vitro cultured protein products of the present disclosure. For instance, in some embodiments, the scaffold may be derived from natural or synthetic (and preferably non-toxic) biomaterials, such as textured vegetable proteins, pectin, flour, collagen, fibronectin, laminin, or other extracellular matrices (e.g., hydrogels). In other embodiments, the scaffold may be derived from synthetic biomaterials, such as hydroxyapatite, alginate, polyglycolic acid, polylactic acid, or their copolymers, including hydrogel preparations derived from these agents. In addition, the scaffolds of the present disclosure may be formed as a solid or semisolid support or a temperature dependent hydrogel (e.g., plant based hydrogels). Commercially available hydrogels, including but not limited to alginate-based hydrogels, may also be used as scaffolds in some embodiments.

The scaffolds of the present disclosure may also have various shapes and structures. For instance, in some embodiments, the scaffold may be a three-dimensional support structure. Scaffold 16 shown in FIG. 2 is an illustrative example of a three-dimensional scaffold that is suitable for various embodiments of the present disclosure.

In more specific embodiments, the scaffold structure may be sculpted into different sizes, shapes, and forms as desired. For instance, the scaffold may be structured to resemble the shape and form of muscle tissues, such as steak, tenderloin, shank, chicken breast, drumstick, lamb chops, fish fillet, lobster tail, and the like.

In additional embodiments, a three-dimensional scaffold may also be molded to include a branched vascular network that provides for delivery of nutrients into and shuttling out of metabolites from the cells at the inner mass of the protein product. In this particular embodiment, the branch vascular network may be edible by using non-toxic natural or synthetic biomaterials as mentioned above. Furthermore, the scaffold may also include adhesion peptides, cell adhesion molecules, or other growth factors covalently or non-covalently associated with the scaffold.

To provide for optimal cell and tissue growth, the scaffolds of the present disclosure preferably have high porosity. Without being bound by theory, it is envisioned that such porous scaffolds can provide maximal surface area for cell attachment.

Stem Cell Differentiation

Various methods may be used to induce the differentiation of stem cells on the above-mentioned scaffolds. For instance, in some embodiments, stem cell differentiation may be induced by adding to the growth medium at least one differentiation-inducing agent. Non-limiting examples of differentiation-inducing agents include sodium butyrate (NaBu), dimethyl sulfoxide (DMSO), 12-O-tetradecanoylphorbol-13-acetate (TPA), retinoic acid (RA), dimethylformamide (DMF), hexamethylene bisacetamide (HMBA), forskolin, and the like.

In other embodiments, stem cell differentiation may be induced by adding to the growth medium at least one differentiation-inhibiting agent, such as lithium chloride or lithium citrate. In additional embodiments, stem cells may be differentiated by adding to the growth medium at least one differentiation-inhibiting agent and at least one differentiation-inducing agent. In further embodiments, the steprn cells may be differentiated by applying a magnetic or a fluid flow field to the growth medium. In further embodiments, the stem cells of the present disclosure may be differentiated by applying a chemo-attractant or an electric field to the scaffold. Other methods of inducing the differentiation of stem cells can also be envisioned by a person of ordinary skill in the art.

A person of ordinary skill in the art will also recognize that various apparatus and systems may be used to induce stem cell differentiation. In some embodiments, and with reference again to FIG. 2, bioreactor 10 may be used to differentiate stem cells 24 on scaffold 16. In such embodiments, electrodes 18, 20 and 22 may also be utilized to apply electric current, oscillating current, or fluidic waves in order to facilitate the differentiation process.

The above-mentioned methods can induce stem cells to differentiate into one or more cell types or tissues. Non-limiting examples of such cell types and tissues include muscle cells, cartilage, connective tissue, blood vessels, and visceral wall tissues.

Without being bound by theory, Applicant also notes that exposing the stem cells or the protein products in vitro to currents or fluidic waves may mimic exercise and increase the similarity in texture between protein product grown in vitro and meat derived from whole animals. The electric or oscillating current may also function to increase the growth and migration rate of the stem cells in vitro. Accordingly, in additional embodiments, the electric or oscillating current may also be applied to the stem cells after differentiation.

Applications

In some embodiments, the methods of the present disclosure may be used to produce protein products that may be used as human food. In additional embodiments, the methods of the present disclosure may be used to produce protein products that may be used as pet food for various animals, such as dogs, cats and fish.

By way of background, the pet food industry is an extension of the human food and agriculture industries. The pet food and commercial livestock and fish feed industries utilize restructured meat chunks mixed with other texture adding ingredients like semi refined carrageenan liquid jelly, other hydrocolloids, and dry ingredients. Moreover, animals frequently require vitamins, conditioning, and various preventative and curative medicinal supplements. These are often difficult and inconvenient to administer, acquire and store. In addition, it is generally difficult to maintain a correct and regular dosage regimen of the above-mentioned supplements for animals.

Additionally, there exists a need for palatable pet food compositions that, in addition to delivering the required nutritional value, would also deliver to the pet beneficial agents such as medicaments, prebiotics, probiotics and the like. Accordingly, the methods of the present disclosure can be used to address such needs.

A person of ordinary skill in the art will also recognize that the methods of the present disclosure may be used in various other settings and for numerous other purposes. For instance, the methods of the present disclosure can be used to produce in-vitro cultured protein products with plant based protein compositions that are enhanced with stem cells of animal origin. In more specific embodiments, the methods of the present disclosure may be used to produce non-human meat products, such as hybrid plant-animal in-vitro protein products with palate-friendly tastes or textures. Additionally, the methods of the present disclosure may be used to provide nutrients to an animal by feeding an animal with in-vitro cultured protein product compositions that were produced by the aforementioned methods.

From the above disclosure, a person of ordinary skill in the art will also recognize that the present invention has numerous embodiments and applications. Reference will now be made to more specific embodiments of the present disclosure. However, Applicant notes that the disclosure below is for exemplary purposes only and is not intended to limit the scope of the claimed invention in any way.

### Example 1

### Isolation of Stem Cells from Organisms

Stem cells (and their niche aggregates) may be isolated from sectioning of the lower 30-90% of an organism with complete regenerative potential. The organism will be attached to the surface of a bioreactor via a foot plate in the lower section of the organism.

The isolated stem cells (and their niche aggregates) will be placed in a nutrient rich medium as described above, along with a 1 mM concentration of lithium chloride or lithium citrate to prevent development of a neural organizer or head structure.

The sectioned regions of the organism with the neural organizer and head structure will be eluted by a fluidic wave into a different bioreactor and bathed in nutrient rich medium as described above, but without any lithium ions. Thus, the sectioned regions will be able to regenerate into complete organisms. Electrical fields may also be used to induce proliferation, migration and differentiation. It is expected that the composition of the membranes of the developing stem cells will change based on the composition of the medium.

After sufficient proliferation, the cells will be mechanically separated into single cells or small clusters of cells. The cells will then be mixed into a homogenous suspension of scaffold, which may be accomplished by mechanical molding of the scaffold, including piercing of the scaffold with such items as needles. Chemo-attractants or electrical fields may be applied to the scaffold to induce the stem cells to migrate into the spaces within the scaffold.

The scaffold, along with embedded stem cells, is then removed from the bioreactors. The scaffold may then be further conditioned, pasteurized, frozen, irradiated or cooked to make it more edible.

### Example 2

### Isolation of Stem Cells from Hyrda

The method described in Example 1 may be used to isolate stem cells from a Hydra species. By way of background, Hydra species have unlimited regenerative capacity. See, e.g., Martinez, D. E. (May 1998), “Mortality patterns suggest lack of senescence in hydra”, Experimental Gerontology 33 (3): 217-225; and Gierer A et al., (September 1972) “Regeneration of hydra from reaggregated cells”, Nat New Biol.; 239 (91):98-101. However, lithium ions, such as lithium chloride or lithium citrate, have been shown to hamper such regenerative capacity. See, e.g., Hassel, M. et al. (1993), “Pattern Formation in Hydra vulgaris is controlled by lithium-sensitive processes.” Developmental Biology 156: 362-371. In addition, Hydra species provide a good source of stems cells, such as epithelial, endothelial, and interstitial stem cells. Furthermore, and as described previously, it is envisioned that Hydra stem cells that are isolated along with their niche aggregates can proliferate more readily than stem cells isolated without the niche aggregates.

Non-limiting examples of Hydra species that may be suitable for use with the methods and compositions of the present disclosure include, without limitation, Hydra americana, Hydra attenuata (or Hydra vulgaris), Hydra canadensis, Hydra carnea, Hydra cauliculata, Hydra circumcincta, Hydra hymanae, Hydra littoralis, Hydra magnipapillata, Hydra minima, Hydra oligactis, Hydra oregona, Hydra pseudoligactis, Hydra rutgerensis, Hydra utahensis, Hydra viridis, and Hydra viridissima.

Referring now to FIG. 3, a depiction of the steps involved in isolating stem cells from Hydra 40 to manufacture stem cell enhanced protein products in accordance with some embodiments of the present disclosure is shown (Applicant notes that Hydra 40 can refer to any of the above-mentioned Hydra species).

In Step 1, Hydra 40 will be grown in large glass containers to which they will attach via a foot plate 42. These organisms will be bathed in growth medium 44 in containers 46 with properties and conditions that were previously described.

Hydra 40 may be cut 2-6 millimeters from the top as a stimulus for the stem cells to replicate. The cutting or sectioning will be performed by an automatic tome 48 designed to section the organisms 2-6 mm from the top (unattached) region. An immobilizing agent may be added to growth medium 44 in order to improve the efficiency of tome 48. The cutting will leave basal sections 40(a) attached to floor plate 42 and release top sections 40(b) into container 46.

In Step II, top sections 40(b) from container 46 will be decanted using fluidic wave or other technology into a second container 50 with the same growth medium 44 as above and permitted to regenerate into new, complete organisms. Regenerated Hydra 40 may then be used again in step 1 as shown. To promote regeneration, growth medium 44 in container 50 will not contain any lithium ions, such as lithium chloride or lithium citrate.

As also shown in Step II, the basal sections 40(a) of Hydra 40 in the first container 46 will be exposed to 1 mM of lithium chloride or lithium citrate in growth medium 44 in order to inhibit regeneration. As a result, sections 40(a) will not grow neural organizers and/or heads.

As shown in Step III, the basal sections 40(a) may again be cut (as in Step 1) to stimulate replication of stem cells within the tubular bases of the headless (and without neural organizer) organism sections. This cycle may be repeated several times.

As shown in Step 1V, once sufficient masses of cells has been generated, they may be harvested and mixed mechanically with specially conditioned scaffolds that were previously described. The scaffolds may then be further processed, flavored or otherwise conditioned into edible food products for fish, birds or other animals (including humans).

From the foregoing detailed description of specific embodiments of the invention, it should be apparent that novel in vitro-cultured protein products and novel methods of making such compositions have been disclosed. Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. It is therefore contemplated that the claims will cover any such modifications or embodiments that fall within the true scope of the invention.

What is claimed is:

1. A method of producing an in-vitro cultured food product, said method comprising:
isolating stem cells from an organism;
culturing said stem cells in a growth medium;
attaching said stem cells to an edible scaffold;
inducing a migration of said stem cells onto said edible scaffold; and
inducing a differentiation of said stem cells into a certain cell type, wherein said in-vitro cultured food product comprises said differentiated stem cells and said scaffold.

2. The method of claim 1, wherein said induction of stem cell differentiation comprises adding to said growth medium at least one differentiation-inducing agent.

3. The method of claim 1, wherein said induction of stem cell differentiation comprises adding to said growth medium at least one differentiation-inhibiting agent.

4. The method of claim 1, wherein said induction of stem cell differentiation comprises applying a magnetic field, an electrical field or a fluid flow field to said stem cells.

5. The method of claim 1, wherein said stem cells differentiate into at least one of muscle cells, cartilage, connective tissue, vascular tissue, nerve tissue, fat tissue, blood vessels, or visceral wall elements.

6. The method of claim 1, wherein said method occurs in a bioreactor.

7. The method of claim 6, wherein said bioreactor houses said edible scaffold and said growth medium.

8. The method of claim 1, wherein said organism is a Hydra species.

9. An in-vitro cultured food product, wherein said composition is produced by steps comprising:
isolating stem cells from an organism;
culturing said stem cells in a growth medium;
attaching said stem cells to an edible scaffold;
inducing a migration of said stem cells onto said edible scaffold; and
inducing a differentiation of said stem cells into a certain cell type, wherein said in-vitro cultured food product comprises said differentiated stem cells and said edible scaffold.

10. The in-vitro cultured food product of claim 9, wherein said organism is a vertebrate.

11. The in-vitro cultured food product of claim 9, wherein said organism is an invertebrate.

12. The in-vitro cultured food product of claim 11, wherein said organism is a Hydra species.

13. The in-vitro cultured food product of claim 9, wherein said stem cells are embryonic stem cells.

14. The in-vitro cultured food product of claim 9, wherein said stem cells are adult stem cells.

15. The in-vitro cultured food product of claim 9, wherein said stem cells are selected from the group consisting of: fibroblasts, myoblasts, epithelial stem cells, endothelial stem cells, interstitial stem cells, stromal cells, mesenchymal stem cells, hematopoietc stem cells, mesangioblasts, and neuroblasts, individually or in combination.

16. The in-vitro cultured food product of claim 9, wherein said stem cells further comprise stem cell-niche aggregates.

17. The in-vitro cultured food product of claim 9, wherein said growth medium comprises an additive.

18. The in-vitro cultured food product of claim 17, wherein said additive inhibits a regeneration of said organism.

19. The in-vitro cultured food product of claim 17, wherein said additive is selected from the group consisting of: bioproteins, vitamins, minerals, amino acids, ribonucleotides, nutrients, drugs, medicaments, prebiotics, probiotics, and antigens, individually or in combination.

20. The in-vitro cultured food product of claim 17, wherein said additive is a flavoring agent.

21. The in-vitro cultured food product of claim 9, wherein said edible scaffold is a three-dimensional scaffold.

22. The in-vitro cultured food product of claim 9, wherein said edible scaffold is selected from the group consisting of: textured vegetable protein, tofu, gluten, pectin, flour, collagen, fibronectin, laminin, extracellular matrices, hydrogels, and synthetic biomaterials, individually or in combination.

23. The in-vitro cultured food product composition of claim 9, wherein said edible scaffold is selected from the group consisting of: hydroxyapatite, alginate, polyglycolic acid, polylactic acid, and copolymers thereof.

24. A method of providing nutrients to an animal, wherein said method comprises feeding said animal the in-vitro cultured food product composition of claim 9.

25. The method of claim 24, wherein said animal is a non-human animal.
